(19) United States
(12) Patent Application Publication
(10) Pub. No.: US 2009/0130637 A1
(43) Pub. Date: May 21, 2009

(54) Tongue scraper

(76) Inventor: Irineo Marvin Pantangco, III, San Diego, CA (US)

Correspondence Address: GORDON & REES LLP, 101 WEST BROADWAY, SUITE 1600, SAN DIEGO, CA, 92101, US

(21) Appl. No.: 11/985,954

(22) Filed: Nov. 19, 2007

(51) Int. Cl. A61B 17/24; A61C 15/00

(52) U.S. Cl. 433/216; 606/161

(57) ABSTRACT

The present invention is a tongue cleaning device that reduces or eliminates the gag reflex during use comprising, a body having a first end and a second end. The first end forming an elongated handle having a contour for ease of handling and use and a gripping means. The second end forming a head having two or more elongated apertures forming at least two parallel ridges perpendicular to the handle. The head being flat and narrow in width and the body being narrow in thickness thereby eliminating or reducing the gag reflex during use.

### CROSS-REFERENCE TO RELATED APPLICATIONS

None

### STATEMENT REGARDING FEDERALLY SPONSORED RESEARCH OR DEVELOPMENT

None

### INCORPORATION-BY-REFERENCE OF MATERIAL SUBMITTED ON COMPACT DISC

None

### BACKGROUND OF THE INVENTION

(1) Field of the Invention

The present invention is a tongue scraper for removing food particles from the tongue. More particularly for reducing or eliminating halitosis, bacteria, tooth decay and increasing oral hygiene.

(2) Description of Related Art

The present invention is a novel tongue scraper for effective oral hygiene. A number of tongue scrapers or cleaning devices have been patented in the past but each have disadvantages. U.S. Pat. No. 2,218,072 to G. D. Runnels discloses a tongue scraper having a handle having a shank and a resilient head affixed to the shank. The resilient head having six ridges perpendicular to the handle with the second, third and fourth ridges having a triangular shaped cross section and the first, fifth and sixth having a square shaped cross section. The head is a solid body made of a flexible material such as rubber with ridges on one side.

The tongue scraper of patent U.S. Pat. No. 2,218,072 has the disadvantages that the ridges are flexible reducing the effectiveness of removing food particles from between the taste buds, the head is a solid body limiting the amount of food particles that can be collected as well as making cleaning of the devise difficult and the head is approximately 2.5 to 3.0 times the width and approximately 1.5 to 2.0 times the thickness which the handle which could cause gagging during use. U.S. Pat. No. 2,583,750 to Runnels is similar to U.S. Pat. No. 2,218,072 but further comprises a resilient neck. This device suffers from the same disadvantages as those described for U.S. Pat. No. 2,218,750 with the added disadvantage that the neck is flexible. Since both these device comprise a handle and a head in the same plane with resilient ridges the addition of a flexible neck further exacerbates the inefficiency of collecting food particles from between the taste buds.

U.S. Pat. No. 5,792,159 discloses a tongue scraper having an elongated handle with a flat broad head and a plurality of cleaning ribs. This device suffers from the disadvantages that the cleaning head is made of a solid body which limits the amount of food particles that can be collected and makes cleaning the devise difficult, the ribs are rounded edge blunt ridges which reduce the effectiveness of removing food particles from between the taste buds and the head is flat and broad being approximately 1.0 to 1.5 times the thickness and 2.5 to 3.0 times the width of the handle which increases the chance of causing a gag reflex by the user.

Consequently there is a need for a tongue cleaner that provides ridges of sufficient rigidity to effectively remove food particles from the tongue, that can be efficiently and easily cleaned and reduces the chance of causing a gag reflex by the user.

### BRIEF SUMMARY OF THE INVENTION

The present invention is a tongue-cleaning device that reduces or eliminates the gag reflex during use. The device comprises a body having a first end and a second end. The first end forms an elongated handle with a gripping means and the second end forms a head having two or more elongated apertures forming at least two parallel ridges perpendicular to the handle. The body is narrow in thickness and the handle is contoured for ease of handling and use. The head is flat and narrow in width thereby eliminating or reducing the gag reflex during use.

In a preferred embodiment the tongue cleaning device is formed in a single piece having a thickness of not less than about 1/32 inch and not greater than about 3/32 inch.

In another preferred embodiment the head has a width of not less than about ⅜ inch and not more than about ⅞ inch.

Another aspect of the present invention comprises methods of reducing or eliminating halitosis, reducing bacteria in the mouth and/or reducing tooth decay utilizing the tongue cleaning device of the present invention. These methods comprise the steps of scraping the tongue with a tongue scraper of the present invention and rinsing the mouth with a solution to remove debris and bacteria loosened or dislodged from the tongue during scraping.

In yet another aspect of the present invention a system for oral hygiene maintenance and a process for maintaining oral hygiene is provided comprising a tooth brush for brushing the teeth, dental floss for removal of food particles from between the teeth after brushing, a tongue scraper of the present invention for removing debris and bacteria from the tongue after brushing and flossing, and a solution for rinsing the mouth after brushing said teeth and scraping said tongue to remove loosened or dislodged debris from the mouth.

### BRIEF DESCRIPTION OF THE SEVERAL VIEWS OF THE DRAWINGS

FIG. 1 Perspective view of one embodiment of the present invention.

FIG. 2 (A) is a top view; (B) is a front view; and (C) is a back view (D) is the bottom view (E) and side view of the tongue scraper of one embodiment of the present invention.

### DETAILED DESCRIPTION OF THE INVENTION

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail.

The term “gripping means” as used herein refers to any means for enhancing the ability of the user to firmly grip the handle of the scraper under dry or wet conditions. Examples include coating the handle with a liquid polymer that dries into a polymer sleeve, affixing a textured sleeve over the handle, providing textured polymer handle pieces that can be inserted into and affixed within apertures or grooves provided in the handle or texturing the surface of the handle.

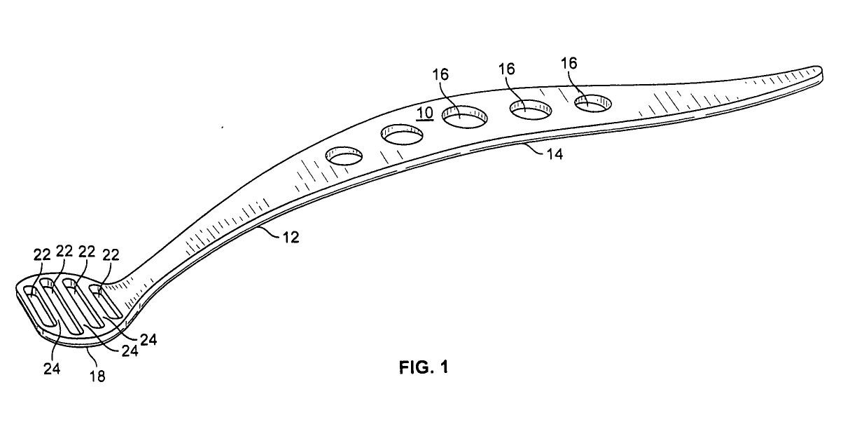

The present invention is a tongue scraper that removes dental plaque, odor causing bacteria, odor causing compounds such as volatile sulfur compounds and food debris including dead epithelial cells, fungi and viruses (see FIG. 1). More specifically, it is a tongue-cleaning device that reduces or eliminates the gag reflex during use comprising, a body having a first end and a second end. The first end forming an elongated handle having a contour for ease of handling and use and a gripping means. The second end forming a head having two or more elongated apertures forming at least two parallel ridges perpendicular to the handle. The head being flat and narrow in width and the body being narrow in thickness thereby eliminating or reducing the gag reflex during use.

One of the major causes of bad breath also known as halitosis is the presence of food particles remaining on the tongue after eating. These particles become trapped between the taste buds and grooves of the tongue. The presence of these particles allows bacteria present in the mouth to survive and propagate. Often times it is these bacteria that either directly or indirectly cause or assist in tooth decay. Consequently, proper oral hygiene is important in maintaining healthy teeth and fresh breath. A tongue scraper can be used to remove residual food particles from the tongue thereby reducing the amount of bacteria in the mouth, which contributes to halitosis and tooth decay. A number of tongue scrapers are available commercially but most if not all do not consider, discuss or disclose how to prevent the common gag reflex that occurs when an object is inserted into the mouth. The present tongue scraper overcomes utilizes effective rigid ribs to remove food particles from the tongue while at the same time providing a narrow low profile head to reduce or eliminate the potential of a gag reflex by the user. In addition the configuration also allows for effective and efficient cleaning to prevent undesirable organisms from growing on the scraper.

### Handle

The scraper body 12 can be provided in one or more pieces that can be assembled by the user or prior to sale. For example, the head 18 and handle 14 may be provided separately for permanent assembly prior to sale. Alternatively, the scraper 10 may be provided in a replaceable head configuration. For example, the head 18 may have a female adapter able to receive a male connector on the handle 14 so that the head 18 may be replaced on a regular basis. Such a configuration is presently utilized on replaceable head razors. Preferably the tongue scraper body 12 of the present invention is provided in a single piece for ease of manufacture and cleaning.

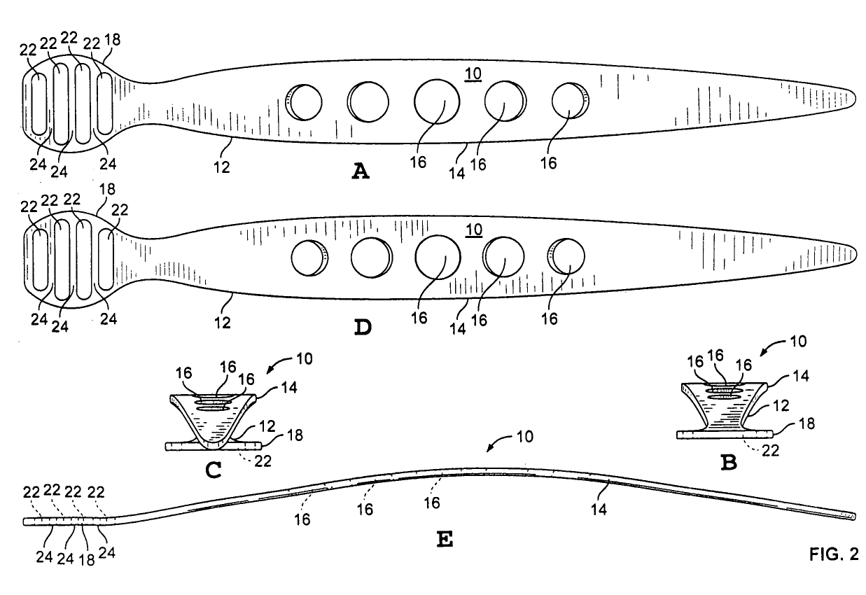

In one particularly preferred configuration a tongue scraper body blank is cut from a desired sheet stock material which is later modified to include apertures 22 that create the ridges 24 for scraping such as by drilling or high pressure water cutting and contoured such as by bending for easy gripping (see FIG. 2). Alternatively, the scraper body 12 may be stamp press cut from stock sheet material with all desired apertures 16 and 22 for later contouring. Correspondingly, the tongue scrapper body 12 may be injection molded having the desired contour and apertures 16 and 22 for creating the scraping ridges 24.

The scraper body 12 may be made of a variety of materials including polymer, plastic or metal. It is preferable that the material selected be able to withstand a variety of cleaning regimens including for example, autoclaving. In view of this, one preferred material is metal with surgical stainless steel being most preferred. The metal may also be treated by applying another metal such as by plating, by physical treatment such as by stamping or brushing or by chemical modification such as surface oxidizing.

The handle 14 can be provided in a number of lengths and widths depending on the size of the hand of the user. For example, a wider longer handle may be provided for an adult such as a width from about ½ to ¾ inch and a length of from about 5 to 8 inches while a thinner shorter handle may be provided for a child such as a width of about ¼ to about ½ inch and a length of 3 to about 6 inches. The handle 14 may be contoured to fit more comfortably in the hand of the user. For example, the handle 14 may be arched slightly to better fit within the palm of the hand. This may be accomplished by form molding the handle 14 during manufacturing or by bending.

The handle 14 may be provided with a gripping means 16 such as a polymer sleeve that slips over the handle 14 or the handle 14 may be dipped into a liquid polymer solution that when dries forms a polymer sleeve over the handle 14, texturing the handle, providing polymer textured pieces that can be inserted into and affixed within apertures 16 or grooves in the handle 14 or shaped apertures cut into the handle 14.

The body 12 of the scraper is prepared from sheet stock having a thickness that allows the scraper 10 to maintain its integrity without bending during use. This thickness will vary depending on the material selected. For example, if the material is plastic or polymer it may require a greater thickness to maintain its integrity than if the scraper 10 were made of metal. In the preferred embodiment the scraper 10 is made of surgical steel having a thickness preferably not less than about 1/32 inch and not more than about 3/32 inch.

### Head

As stated above the scraper body 12 can be provided in one or more pieces that can be assembled by the user or prior to sale. Further the scraper 10 may be provided in a replaceable head configuration such as those used by commercially available replaceable head razors. However, in a preferred configuration the tongue scraper body 12 is manufactured in a single piece for ease of production and cleaning.

The head 18 is oriented in the same plane as the handle 14 and is provided with two or more elongated apertures 22 that are positioned perpendicular to the handle 14. These apertures are in such proximity to each other to form three or more parallel scraping ridges 24 that are also perpendicular to the handle 14. The apertures 22 may be cut to produce a 90° angle on the scraping ridges 24 so that the ridge shows a square cross-cut profile. Alternatively the aperture 22 may be cut so that the ridge 24 shows a triangle cross-cut profile providing a more tapered edge for cleaning the tongue.

These apertures 22 do not restrict the amount of food particles that can be collected since they do not form pockets. The apertures 22 can easily overfill onto the top of the head 18 for easy removal by washing the head 18 under a faucet.

The head 18 may be oriented at an angle to the handle 14 so that it may be easily positioned relatively parallel to the tongues' surface during cleaning. This bend may be provided angling the head 18 upward above the top surface of the handle 14 or downward toward the bottom surface of the handle 14. The angle may be only a few degrees or may be several degrees. Preferably, the head 18 is angled at about 3 degrees to about 25 degrees.

The head 18 can be provided in a variety of shapes and sizes provided that it maintains its integrity and does not bend and its width and thickness do not cause a gag reflex during use. In order to reduce the possibility of this reflex occurring during use the head 18 width is preferably not less than about ⅜ inch and not more than about ⅞ inch with the thickness being not less than about 1/32 inch and not more than about 3/32 inch.

### Use

During use the tongue scraper is positioned firmly in the hand and inserted into the mouth. The head is placed flat against the tongue and dragged across the tongue from the back to the front of the tongue. To avoid reintroducing food particles present on the scraper rinse the head of the scraper under warm water and duplicate the process until all or a majority of the tongue has been scrapped. When scraping is completed it is recommended to wash the scraper thoroughly and if possible place the scrapper in a solution of antibacterial solution such as mouth wash until its next use. Periodic high temperature cleaning is also recommended such as by autoclave or by placing the scraper in the dish washer during a normal full length cycle.

Removal of food particles will reduce the nutrients available for bacteria to propagate in the mouth. This will in turn reduce the onset of chronic halitosis and improve general breath odor as well as reduce bacterial induced tooth decay. Consequently a good oral hygiene program should include tongue scraping on a regular basis along with brushing the teeth, the use of dental floss, mouth wash and regular dental check ups.

1. A tongue cleaning device that reduces or eliminates the gag reflex during use comprising;
a body having a first end and a second end said body having a narrow thickness; said first end forming an elongated handle said handle having a contour for ease of handling and use and gripping means; said second end of said body forming a head having two or more apertures said apertures being elongated forming at least two parallel ridges perpendicular to said handle, said head being flat and narrow in width thereby eliminating or reducing the gag reflex during use.

2. A tongue cleaning device according to claim 1 wherein said body is formed in a single piece having a thickness of not less than about 1/32 inch.

3. A tongue cleaning device according to claim 1 wherein said body is formed in a single piece having a thickness of not greater than about 3/32 inch.

4. A tongue cleaning device according to claim 1 wherein said head has a width of not less than about ⅜ inch.

5. A tongue cleaning device according to claim 1 wherein said head has a width of not more than about ⅞ inch.

6. A method of reducing or eliminating halitosis utilizing a tongue cleaning device comprising the steps of:
scraping the tongue with a tongue scraper comprising a body having a first end and a second end said body having a narrow thickness; said first end forming an elongated handle said handle having a contour for ease of handling and use and gripping means; said second end of said body forming a head having two or more apertures said apertures being elongated forming at least two parallel ridges perpendicular to said handle, said head being flat and narrow in width thereby eliminating or reducing the gag reflex during use and
rinsing the mouth with a solution to remove debris and bacteria loosened or dislodged from the tongue during scraping.

7. A method of reducing bacteria in the mouth by utilizing a tongue cleaning device comprising the steps of:
scraping the tongue with a tongue scraper comprising a body having a first end and a second end said body having a narrow thickness; said first end forming an elongated handle said handle having a contour for ease of handling and use and gripping means; said second end of said body forming a head having two or more apertures said apertures being elongated forming at least two parallel ridges perpendicular to said handle, said head being flat and narrow in width thereby eliminating or reducing the gag reflex during use and
rinsing the mouth with a solution to remove bacteria dislodged from the tongue during scraping but not captured by said scraper.

8. A method of reducing tooth decay by utilizing a tongue cleaning device comprising the steps of:
scraping the tongue with a tongue scraper comprising a body having a first end and a second end said body having a narrow thickness; said first end forming an elongated handle said handle having a contour for ease of handling and use and gripping means; said second end of said body forming a head having two or more apertures said apertures being elongated forming at least two parallel ridges perpendicular to said handle, said head being flat and narrow in width thereby eliminating or reducing the gag reflex during use and
rinsing the mouth with a solution to remove bacteria dislodged from the tongue during scraping but not captured by said scraper.

9. A system for oral hygiene maintenance comprising: a tooth brush for brushing the teeth;
dental floss for removal of food particles from between the teeth after brushing;
a tongue scraper for removing debris and bacteria from the tongue after brushing and flossing said tongue scraper comprising a body having a first end and a second end said body having a narrow thickness; said first end forming an elongated handle said handle having a contour for ease of handling and use and gripping means; said second end of said body forming a head having two or more apertures said apertures being elongated forming at least two parallel ridges perpendicular to said handle, said head being flat and narrow in width thereby eliminating or reducing the gag reflex during use; and
a solution for rinsing the mouth after brushing said teeth and scraping said tongue to remove loosened or dislodged debris from the mouth.

10. A process for maintaining oral hygiene comprising:
brushing the teeth with a tooth brush;
dental floss for removal of food particles from between the teeth after brushing;
scraping the tongue with a tongue scraper comprising a body having a first end and a second end said body having a narrow thickness; said first end forming an elongated handle said handle having a contour for ease of handling and use and gripping means; said second end of said body forming a head having two or more apertures said apertures being elongated forming at least two parallel ridges perpendicular to said handle, said head being flat and narrow in width thereby eliminating or reducing the gag reflex during use; and
rinsing the mouth with a solution after brushing said teeth and scraping said tongue to remove loosened or dislodged debris from said mouth thereby maintaining oral hygiene.
